(19) United States
(12) Patent Application Publication
(10) Pub. No.: US 2017/0281895 A1
(43) Pub. Date: Oct. 5, 2017

(54) COUPLING AND SUPPORTING OF APPARATUS ON THE FACE AND NECK

(71) Applicant: Venti-Lock Systems LLC, University Heights, OH (US)

(72) Inventors: Joel Kessler, Efrat (IL); Fred Kessler, Efrat (IL); Ari Isaacs, Oceanside, NY (US)

(73) Assignee: Venti-Lock Systems LLC, University Heights, OH (US)

(21) Appl. No.: 15/508,309

(22) PCT Filed: Sep. 2, 2015
(86) PCT No.: PCT/IL2015/050883

§ 371 Date: Mar. 2, 2017

Related U.S. Application Data
(60) Provisional application No. 62/044,622, filed on Sep. 2, 2014.

(51) Int. Cl. A61M 16/06; A61M 16/10; A61M 16/04

(52) U.S. Cl. CPC A61M 16/0688; A61M 16/0497; A61M 16/105; A61M 16/0672

(57) ABSTRACT

Apparatus (58) is provided for coupling a cannula (59) to a face of a subject (24), the apparatus (58) comprising an adhesive pad (60) configured to adhere to a surface of the subject's face, and one or more cannula grips (62) rotatably coupled to the adhesive pad (60) and configured to couple the cannula (59) to the subject's face by gripping the cannula (59). Other embodiments are also described.

### CROSS-REFERENCE TO RELATED APPLICATIONS

The present application claims priority from U.S. Provisional Application 62/044,622, filed Sep. 2, 2014, which is assigned to the assignee of the present application and is incorporated herein by reference.

### FIELD OF THE INVENTION

The present invention relates generally to coupling medical apparatus to a face of a subject, and to supporting medical apparatus over a portion of a subject's body.

### BACKGROUND

U.S. Pat. No. 6,328,038 to Kessler, which is incorporated herein by reference, describes a nasal cannula retainer having a flexible member and cannula grips. The flexible member has a central nose portion with an adhesive backing for adhering to a patient's nose, and two cheek portions connected to the nose portion, each of the cheek portions having an adhesive backing for adhering to a patient's cheek. The adhesive layers are covered with a peel layer which is removed prior to applying the retainer. The cannula grips are attached to the cheek portions to secure a nasal cannula on each of the patient's cheeks. The grips are releasable and reusable, allowing the cannula to be repositioned or temporarily removed without the need for the retainer to be discarded and replaced, and without the use of tape to secure the cannula to the retainer after the cannula is repositioned. Other embodiments are also described.

### SUMMARY OF THE INVENTION

When coupling medical apparatus to a face of a subject, and when supporting medical apparatus over a portion of a subject's body, it is generally desired to reduce, to the extent possible, mechanical forces (e.g., pressure, tension, or rubbing) applied to the subject's skin.

Some applications of the present invention include apparatus for coupling a mask to a face of a subject, such as an oxygen or filter mask. The apparatus includes two adhesive pads configured to adhere to a surface of the subject's face, each of the pads having a surface area of between 1 and 50 cm2. Two straps are coupleable at first ends of the straps to the adhesive pads, respectively, and, at respective second ends of the straps, opposite the first ends, are shaped so as to define respective rotation couplings, which are rotatably coupleable to the coupling holes of the mask, respectively. Each strap has a length of between 2 and 40 cm.

Some applications of the present invention also include apparatus for supporting a tracheostomy-tube holder over a tracheostomy. The apparatus includes a first support and a second support. The supports include respective adhesive pads, and respective straps, each having a length between 1 and 40 cm and a width between 0.25 and 3 cm, which are configured to be coupled to the tracheostomy-tube holder and the adhesive pads, respectively.

Some applications of the present invention also include apparatus for coupling a cannula to a face of a subject. The apparatus includes an adhesive pad configured to adhere to a surface of the subject's face, and at least one cannula grip rotatably coupled to the pad. The cannula grip is configured to couple the cannula to the subject's face by gripping the cannula.

There is therefore provided, in accordance with an application of the present invention, apparatus for coupling a cannula to a face of a subject, the apparatus including:

an adhesive pad configured to adhere to a surface of the subject's face; and

one or more cannula grips rotatably coupled to the adhesive pad and configured to couple the cannula to the subject's face by gripping the cannula.

For some applications, each of the one or more cannula grips is shaped to define two gripping arms configured to grip the cannula therebetween, a distance between the gripping arms being between 2 and 10 mm.

For any of the cannula applications described herein, the apparatus may further include the cannula.

For some applications, the adhesive pad has a surface area between 2 and 10 cm2, such as between 2 and 5 cm2.

For some applications, the apparatus includes two adhesive pads, which are configured to adhere to respective cheeks of the subject, and which have respective surface areas that are less than 10 cm2.

For some applications, the apparatus includes two cannula grips, which are rotatably coupled to the adhesive pad and configured to couple the cannula to the subject's face by gripping the cannula.

For some applications, the adhesive pad is shaped so as to define a weakened line to facilitate splitting of the adhesive pad into two pieces, which are configured to adhere to respective cheeks of the subject; and the two cannula grips are rotatably coupled to the two pieces, respectively. For some applications, the weakened line is perforated.

For some applications, the adhesive pad includes a visual indicator demarcating the adhesive pad into two pieces, which are configured to adhere to respective cheeks of the subject; and the two cannula grips are rotatably coupled to the two pieces, respectively.

For some applications, the apparatus includes two adhesive pads, which are configured to adhere to respective cheeks of the subject, and which have respective surface areas that are less than 10 cm2; and the two cannula grips are rotatably coupled to the two adhesive pads, respectively.

There is further provided, in accordance with an application of the present invention, a method for coupling a cannula to a face of a subject, the method including:

adhering an adhesive pad to a surface of the subject's face; and

coupling the cannula to the subject's face by gripping the cannula with one or more cannula grips that are rotatably coupled to the adhesive pad.

For some applications, each of the one or more cannula grips is shaped to define two gripping arms configured to grip the cannula therebetween, a distance between the gripping arms being between 2 and 10 mm, and coupling the cannula to the subject's face includes gripping the cannula with the gripping arms.

For some applications, the adhesive pad has a surface area between 2 and 10 cm2, such as between 2 and 5 cm2.

For some applications, gripping the cannula with the one or more cannula grips includes gripping the cannula with the one or more cannula grips while rotating the one or more cannula grips. For some applications, gripping the cannula with the one or more cannula grips includes gripping the cannula with the one or more cannula grips before rotating the one or more cannula grips. For some applications, gripping the cannula with the one or more cannula grips includes gripping the cannula with the one or more cannula grips after rotating the one or more cannula grips. For some applications, coupling the cannula to the subject's face includes coupling the cannula to the subject's face after adhering the adhesive pad to the surface of the subject's face. For some applications, adhering the adhesive pad to the surface of the subject's face includes adhering the adhesive pad to a surface of a nose of the subject. For some applications, adhering the adhesive pad to a surface of a nose of the subject includes adhering exactly one adhesive pad to the surface of the nose of the subject.

For some applications:

the one or more cannula grips include two cannula grips, which are rotatably coupled to the adhesive pad and configured to couple the cannula to the subject's face by gripping the cannula, and

adhering the exactly one adhesive pad to the surface of the subject's face includes:
adhering a central portion of the exactly one adhesive pad to the surface of the nose; and
adhering end portions of the exactly one adhesive pad to respective cheeks of the subject, wherein the end portions include the two cannula grips, respectively.

For some applications, adhering the adhesive pad to the surface of the subject's face includes adhering two adhesive pads to respective cheeks of the subject, without adhering the adhesive pads to a nose of the subject.

For some applications, the two adhesive pads have respective surface areas that are less than 10 cm2.

For some applications, coupling the cannula to the subject's face includes gripping the cannula with two cannula grips that are rotatably coupled to the adhesive pad.

For some applications, adhering the adhesive pad to the surface of the subject's face includes:

splitting the adhesive pad into two pieces, wherein the two cannula grips are rotatably coupled to the two pieces, respectively; and

adhering the two pieces at least partially to respective cheeks of the subject.

For some applications, adhering the two pieces at least partially to respective cheeks of the subject includes maintaining the two pieces generally in a same rotational relationship with each other after splitting the adhesive pad into the two pieces, such that respective portions of the two pieces are adhered to respective sides of a nose of the subject, and other respective end portions of the two pieces, which include the two cannula grips, respectively, are adhered to the respective cheeks of the subject.

For some applications, adhering the two pieces at least partially to respective cheeks of the subject includes rotating the two pieces until respective split edges of the two pieces face generally away from each other, and subsequently adhering the two pieces to the respective cheeks of the subject, without adhering the two pieces to a nose of the subject.

For some applications, the adhesive pad is shaped so as to define a weakened line to facilitate splitting of the adhesive pad into two pieces, and splitting the adhesive pad into the two pieces includes splitting the adhesive pad into the two pieces along the weakened line. For some applications, the weakened line is perforated.

For some applications, splitting the adhesive pad into the two pieces includes cutting the adhesive pad into the two pieces. For some applications, the adhesive pad includes a visual indicator demarcating the adhesive pad into the two pieces, and splitting the adhesive pad into the two pieces includes cutting the adhesive pad along the visual indicator.

For some applications, adhering the adhesive pad to the surface of the subject's face includes adhering two adhesive pads to respective cheeks of the subject, the two adhesive pads have respective surface areas that are less than 10 cm2, and the two cannula grips are rotatably coupled to the two adhesive pads, respectively.

There is still further provided, in accordance with an application of the present invention, apparatus for coupling a mask to a face of a subject, the mask shaped so as to define two coupling holes, the apparatus including:

two adhesive pads configured to adhere to a surface of the subject's face, each of the adhesive pads having a surface area between 1 and 50 cm2; and

two straps, each of which has a length of between 2 and 40 cm, and which (a) at respective first ends of the straps, are coupleable to the adhesive pads, respectively, and (b) at respective second ends of the straps, opposite the first ends, are shaped so as to define respective rotation couplings, which are rotatably coupleable to the coupling holes of the mask, respectively.

For some applications, the rotation couplings are shaped so as to define respective head portions and respective neck portions that are adjacent to and narrower than the head portions.

For some applications, the length of each of the straps is between 5 and 15 cm.

For some applications, the two adhesive pads are configured to adhere to respective cheeks of the subject, and have respective lengths that are less than 10 cm.

For some applications, the straps are elastic straps.

For some applications, the surface area of each of the adhesive pads is between 5 and 50 cm2, such as between 15 and 35 cm2.

For some applications, each of the straps is coupleable to a respective one of the adhesive pads so as to provide an adjustable total length of the combined strap and adhesive pad. For some applications, an end the adhesive pad is shaped so as to define a tab, which is sized so as to be passable through a slit defined by the strap near a first end of the strap.

For some applications, the straps are rotatably coupled to the adhesive pads, respectively.

For some applications, each of the straps and respective adhesive pads are configured so as to provide an adjustable total length of the combined strap and adhesive pad.

For any of the applications described above, the mask may be an oxygen mask, and the respective rotation couplings are rotatably coupleable to the coupling holes of the oxygen mask, respectively.

For any of the applications described above, the mask may be a filter mask, and the respective rotation couplings are rotatably coupleable to the coupling holes of the filter mask, respectively.

For any of the mask applications described herein, the apparatus may further include the mask. For some of these applications, the mask is an oxygen mask or a filter mask.

There is additionally provided, in accordance with an application of the present invention, a method for coupling a mask to a face of a subject, the mask shaped so as to define two coupling holes, the method including:

adhering two adhesive pads to a surface of the subject's face, each of the adhesive pads having a surface area between 1 and 50 cm2; and

coupling the mask to the subject's face by:
coupling respective first ends of two straps to the adhesive pads, respectively, each of which straps has a length between 2 and 40 cm, and
rotatably coupling respective rotation couplings to the coupling holes of the mask, respectively, wherein the two straps, at respective second ends of the straps, opposite the first ends, are shaped so as to define the respective rotation couplings.

For some applications, adhering the two adhesive pads to the surface of the subject's face includes adhering the two adhesive pads to the surface of the subject's face before coupling the mask to the subject's face.

For some applications, the rotation couplings are shaped so as to define respective head portions and respective neck portions that are adjacent to and narrower than the head portions.

For some applications, the length of each of the straps is between 5 and 15 cm.

For some applications, the straps are elastic straps.

For some applications, coupling the mask to the subject's face includes coupling the mask to the subject's face such that no portion of the mask touches a nose of the subject.

For some applications, adhering the two adhesive pads to the surface of the subject's face includes adhering the two adhesive pads to respective cheeks of the subject, without adhering the adhesive pads to a nose of the subject.

For some applications, the surface area of each of the adhesive pads is between 5 and 50 cm2, such as between 15 and 35 cm2.

For some applications, each of the straps is coupleable to a respective one of the adhesive pads so as to provide an adjustable total length of the combined strap and adhesive pad, and coupling each of the first ends of the two straps to the respective adhesive pad includes adjusting the total length of the combined strap and adhesive pad. For some applications, an end the adhesive pad is shaped so as to define a tab, and coupling each of the respective first ends of the two straps to the adhesive pads, respectively, includes passing the tab through a slit defined by the strap near a first end of the strap.

For some applications, coupling the straps to the adhesive pads, respectively, includes rotatably coupling the straps to the adhesive pads, respectively.

For some applications, coupling each of the first ends of the two straps to the respective adhesive pad includes coupling each of the respective first ends of the two straps to the respective adhesive pad so as to provide an adjustable total length of the combined strap and adhesive pad.

For some applications, the mask is an oxygen mask, and rotatably coupling the respective rotation couplings to the coupling holes of the mask, respectively, includes rotatably coupling respective rotation couplings to the coupling holes of the oxygen mask, respectively.

For some applications, the mask is a filter mask, and rotatably coupling the respective rotation couplings to the coupling holes of the mask, respectively, includes rotatably coupling respective rotation couplings to the coupling holes of the filter mask, respectively.

There is yet additionally provided, in accordance with an application of the present invention, apparatus for supporting a tracheostomy-tube holder over a tracheostomy, the apparatus including:

first and second supports, including:
respective adhesive pads; and
respective straps, each having a length between 1 and 40 cm and a width between 0.25 and 3 cm, which are configured to be coupled to the tracheostomy-tube holder and the adhesive pads, respectively.

For some applications, the tracheostomy-tube holder is shaped so as to define first and second slots, and the straps are configured to be coupled to the tracheostomy-tube holder by passing the respective straps through first and second slots, respectively.

For some applications, the straps are configured to be coupled to the tracheostomy-tube holder so as to provide leeway for the straps to move with respect to the tracheostomy-tube holder.

For some applications, each strap has a length between 5 and 20 cm. For some applications, each strap has a width between 0.5 and 2 cm. For some applications, each adhesive pad has a surface area of 5-15 cm2.

For some applications, each of the straps is coupleable to a respective one of the adhesive pads so as to provide an adjustable total length of the combined strap and adhesive pad. For some applications, an end the adhesive pad is shaped so as to define a tab, which is sized so as to be passable through a slit defined by the strap near a first end of the strap.

For some applications, the straps are rotatably coupled to the adhesive pads, respectively.

For any of the tracheostomy-tube holder applications described herein, the apparatus may further include the tracheostomy-tube holder. For some of these applications, the apparatus further includes a tracheostomy tube, and the tracheostomy-tube holder is configured to hold the tracheostomy tube.

There is also provided, in accordance with an application of the present invention, a method for supporting a tracheostomy-tube holder over a tracheostomy of a subject, the method including:

adhering respective adhesive pads of first and second supports to a neck of the subject; and

coupling respective straps to the tracheostomy-tube holder and the adhesive pads, respectively, each of the straps each having a length between 1 and 40 cm and a width between 0.25 and 3 cm.

For some applications, coupling the respective straps to the tracheostomy-tube holder includes passing the respective straps through first and second slots, respectively, defined by tracheostomy-tube holder.

For some applications, passing the respective straps through the first and the second slots, respectively, includes passing the respective straps through the first and the second slots, respectively, so as to provide leeway for the straps to move with respect to the tracheostomy-tube holder.

For some applications, each strap has a length between 5 and 20 cm. For some applications, each strap has a width between 0.5 and 2 cm. For some applications, each adhesive pad has a surface area of 5-15 cm2.

For some applications, each of the straps is coupleable to a respective one of the adhesive pads so as to provide an adjustable total length of the combined strap and adhesive pad, and coupling each of the respective straps to the adhesive pad, respectively, includes adjusting the total length of the combined strap and adhesive pad. For some applications, an end the adhesive pad is shaped so as to define a tab, and coupling each of the respective straps to the adhesive pad, respectively, includes passing the tab through a slit defined by the strap near a first end of the strap.

For some applications, coupling the straps to the adhesive pads, respectively, includes rotatably coupling the straps to the adhesive pads, respectively.

There is also provided, in accordance with some applications of the present invention, apparatus for coupling an oxygen mask to a face of a subject, the apparatus including:

at least one adhesive pad configured to adhere to a surface of the subject's face, the pad having a surface area between 2 and 100 cm2; and

a plurality of straps coupled to the pad and configured to couple the mask to the subject's face by looping around a portion of the mask, each strap having a length of between 2 and 40 cm.

In some applications, each strap has a length between 5 and 15 cm.

In some applications, a strip is coupled to an outside surface of the mask, and the straps are configured to couple the mask to the subject's face by looping around the strip.

In some applications, the mask is shaped to define one or more holes, and the straps are configured to couple the mask to the subject's face by passing through the holes.

In some applications, the at least one adhesive pad includes two adhesive pads, which are configured to adhere to respective cheeks of the subject, and which have respective surface areas that are less than 50 cm2. In some applications, the at least one adhesive pad includes two adhesive pads, which are configured to adhere to respective cheeks of the subject, and which have respective lengths that are less than 10 cm.

In some applications, the at least one adhesive pad includes:

a first adhesive-pad portion, shaped to adhere to both cheeks of the subject; and

a second adhesive-pad portion extending from a middle of the first adhesive-pad portion in a direction that is substantially perpendicular to a line joining respective ends of the first adhesive-pad portion.

In some applications, the pad has a surface area between 10 and 100 cm2. In some applications, the pad has a surface area between 30 and 70 cm2. In some applications, the straps are elastic straps. In some applications, respective surfaces at respective distal portions of the straps are harder than respective other portions of the straps.

In some applications, for each of the plurality of straps, a distal portion of the strap is attachable to a proximal portion of the strap even without being tied to the proximal portion. In some applications, the apparatus further includes a coupler, and, for each of the plurality of straps, the distal portion of the strap is attachable to the proximal portion of the strap via the coupler. In some applications, the coupler is selected from the group consisting of: a hook-and-loop, a snap-fastener, a button-and-buttonhole, an adhesive, and a protrusion-coupler. In some applications, for each of the plurality of straps, the distal portion of the strap is attachable to the proximal portion of the strap via an action selected from the group consisting of: a clipping action, a clamping action, and a stapling action.

There is further provided, in accordance with some applications of the present invention, a method for coupling an oxygen mask to a face of a subject, the method including:

adhering at least one adhesive pad to a surface of the subject's face; and

coupling the mask to the subject's face by looping a plurality of straps, which are coupled to the pad, around a portion of the mask.

In some applications, a strip is coupled to an outside surface of the mask, and looping the plurality of straps around the portion of the mask includes looping the plurality of straps around the strip. In some applications, the mask is shaped to define one or more holes, and looping the plurality of straps around the portion of the mask includes passing the straps through the holes. In some applications, coupling the mask to the subject's face includes coupling the mask to the subject's face such that no portion of the mask touches a nose of the subject. In some applications, adhering the at least one adhesive pad to the surface of the subject's face includes adhering the at least one adhesive pad to a surface of a nose of the subject.

In some applications, adhering the at least one adhesive pad to the surface of the subject's face includes adhering two adhesive pads to respective cheeks of the subject, without adhering the pads to a nose of the subject.

In some applications, coupling the mask to the subject's face further includes attaching respective distal portions of the straps to respective proximal portions of the straps, without tying the straps. In some applications, attaching the respective distal portions of the straps to the respective proximal portions of the straps includes using a coupler selected from the group consisting of: a hook-and-loop, a snap-fastener, a button-and-buttonhole, an adhesive, a protrusion-coupler, a clip, a clamp, and a stapler.

There is still further provided, in accordance with some applications of the present invention, apparatus for supporting a tracheostomy-tube holder over a tracheostomy, the apparatus including:

a first support and a second support, each support including:
an adhesive pad;
a strap having a length between 1 and 40 cm and a width between 0.25 and 3 cm; and
a strap-fastener rotatably coupled to the adhesive pad, and coupled to a proximal portion of the strap.

In some applications, each strap has a length between 5 and 20 cm. In some applications, each strap has a width between 0.5 and 2 cm.

In some applications, respective distal portions of the straps are attachable to respective proximal portions of the straps, even without being tied to the respective proximal portions. In some applications, the apparatus further includes respective first and second couplers, and the respective distal portions of the straps are attachable to the respective proximal portions of the straps via the respective couplers. In some applications, each of the respective couplers is selected from the group consisting of: a hook-and-loop, a snap-fastener, a button-and-buttonhole, an adhesive, and a protrusion-coupler. In some applications, the respective distal portions of the straps are attachable to the respective proximal portions of the straps via at least one action selected from the group consisting of: a clipping action, a clamping action, and a stapling action.

In some applications, each adhesive pad has a surface area of 5-15 cm2.

There is additionally provided, in accordance with some applications of the present invention, a method for supporting a tracheostomy-tube holder over a tracheostomy of a subject, the tracheostomy-tube holder having a first slot therein and a second slot therein, the method including:

adhering respective adhesive pads to a neck of the subject;

rotating respective strap-fasteners that are rotatably coupled to the respective adhesive pads; and

passing respective straps that are coupled to the respective strap-fasteners through the first slot and the second slot.

In some applications, the method further includes, after passing the respective straps through the first slot and the second slot, attaching respective distal portions of the respective straps to respective proximal portions of the respective straps.

In some applications, attaching the respective distal portions of the respective straps to the respective proximal portions of the respective straps includes using at least one coupler selected from the group consisting of: a hook-and-loop, a snap-fastener, a button-and-buttonhole, an adhesive, a protrusion-coupler, a clip, a clamp, and a stapler.

In some applications, passing the respective straps through the first slot and the second slot includes passing the respective straps through the first slot and the second slot while rotating the respective strap-fasteners. In some applications, passing the respective straps through the first slot and the second slot includes passing the respective straps through the first slot and the second slot before rotating the respective strap-fasteners. In some applications, passing the respective straps through the first slot and the second slot includes passing the respective straps through the first slot and the second slot after rotating the respective strap-fasteners.

There is yet additionally provided, in accordance with some applications of the present invention, apparatus for coupling a cannula to a face of a subject, the apparatus including:

an adhesive pad configured to adhere to a surface of the subject's face; and

at least one cannula grip rotatably coupled to the pad and configured to couple the cannula to the subject's face by gripping the cannula.

In some applications, the adhesive pad has a surface area between 2 and 10 cm2.

In some applications, the adhesive pad has a surface area between 2 and 5 cm2. In some applications, the at least one adhesive pad includes two adhesive pads, which are configured to adhere to respective cheeks of the subject, and which have respective surface areas that are less than 10 cm2. In some applications, the cannula grip is shaped to define two gripping arms configured to grip the cannula therebetween, a distance between the gripping arms being between 2 and 10 mm.

There is also provided, in accordance with some applications of the present invention, a method for coupling a cannula to a face of a subject, the method including:

adhering an adhesive pad to a surface of the subject's face; and

coupling the cannula to the subject's face by:
rotating at least one cannula grip that is rotatably coupled to the adhesive pad; and
gripping the cannula with the cannula grip.

In some applications, gripping the cannula with the cannula grip includes gripping the cannula with the cannula grip while rotating the at least one cannula grip. In some applications, gripping the cannula with the cannula grip includes gripping the cannula with the cannula grip before rotating the at least one cannula grip. In some applications, gripping the cannula with the cannula grip includes gripping the cannula with the cannula grip after rotating the at least one cannula grip.

In some applications, adhering the at least one adhesive pad to the surface of the subject's face includes adhering the at least one adhesive pad to a surface of a nose of the subject. In some applications, adhering the at least one adhesive pad to the surface of the subject's face includes adhering two adhesive pads to respective cheeks of the subject, without adhering the pads to a nose of the subject.

The present invention will be more fully understood from the following detailed description of applications thereof, taken together with the drawings, in which:

### BRIEF DESCRIPTION OF THE DRAWINGS

FIGS. 1A-B are schematic illustrations of apparatus for coupling an oxygen mask to a face of a subject, in accordance with some applications of the present invention;

FIG. 2 is a schematic illustration of the apparatus of FIGS. 1A-B coupled to the face of the subject, in accordance with some applications of the present invention;

FIG. 3 is a schematic illustration of an attachment of a strap to an adhesive pad of the apparatus of FIGS. 1A-B, in accordance with an application of the present invention;

FIGS. 4A-B are schematic illustrations of apparatus for supporting a tracheostomy-tube holder over a tracheostomy of a subject, in accordance with some applications of the present invention;

FIGS. 5A-B are schematic illustrations of the apparatus of FIGS. 4A-B coupled to the neck of the subject, in accordance with some applications of the present invention;

FIGS. 6A-C are schematic illustrations of attachment of a strap to an adhesive pad of the apparatus of FIGS. 4A-B, in accordance with respective applications of the present invention;

FIG. 7 is a schematic illustration of apparatus for coupling a cannula to a face of a subject, in accordance with some applications of the present invention;

FIG. 8 is a schematic illustration of the apparatus of FIG. 7 attached to the face of the subject, in accordance with an application of the present invention;

FIGS. 9A-B are schematic illustrations of splitting of an adhesive pad of the apparatus of FIG. 7 into two pieces, in accordance with an application of the present invention;

FIG. 10 is a schematic illustration of the pieces of the adhesive pad of FIGS. 9A-B adhered to a surface of the subject's face, in accordance with an application of the present invention;

FIG. 11 is a schematic illustration of rotation of the pieces of the adhesive pad of FIGS. 9A-B, in accordance with an application of the present invention; and

FIG. 12 is a schematic illustration of the pieces of the adhesive pad of FIGS. 9A-B adhered to a surface of the subject's face after being rotated as described with reference to FIG. 11, in accordance with an application of the present invention.

### DETAILED DESCRIPTION OF APPLICATIONS

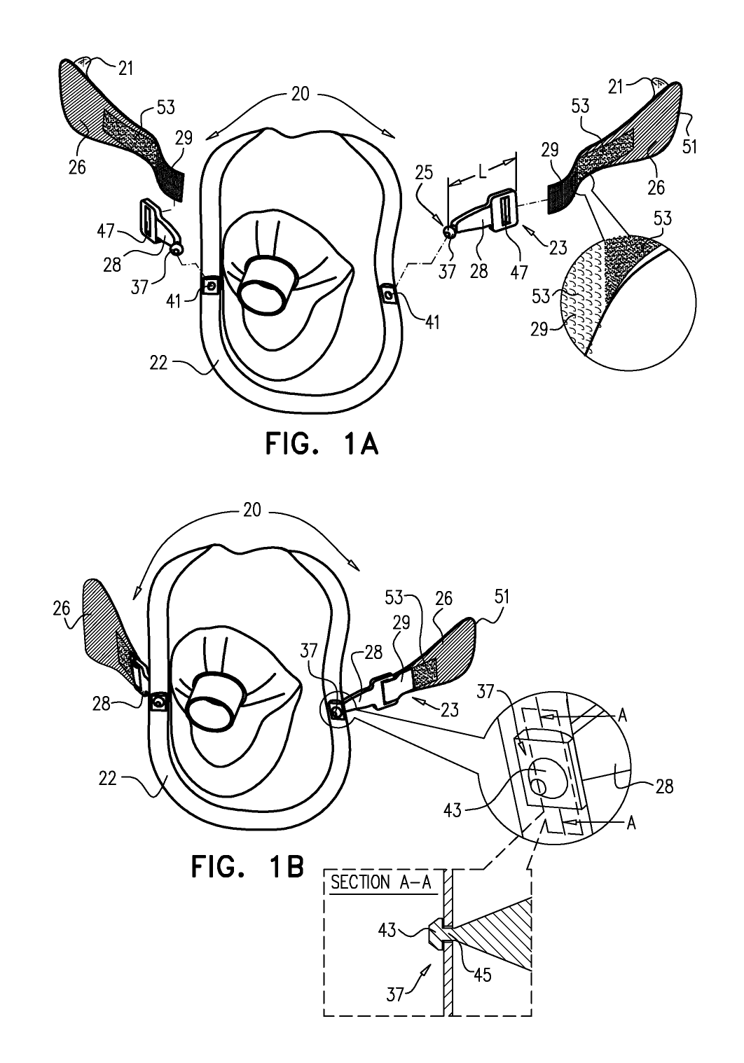

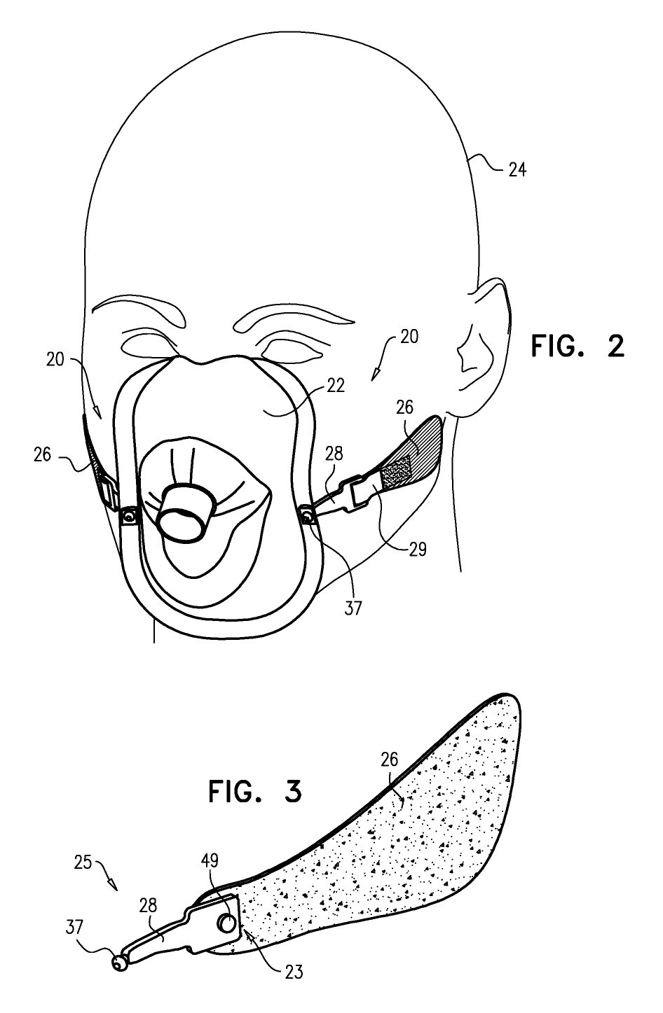

Reference is made to FIGS. 1A-B, which are schematic illustrations of apparatus 20 for coupling an oxygen mask 22 to a face of a subject 24, and to FIG. 2, which is a schematic illustration of apparatus 20 coupled to the face of subject 24, in accordance with an application of the present invention. Apparatus 20 comprises one or more adhesive pads 26 (typically exactly two adhesive pads 26) configured to adhere to a surface of the subject's face, each of the adhesive pads having a surface area that is at least 1 cm2 and/or less than 50 cm2, e.g., between 5 and 50 cm2, such as between 15 and 35 cm2. (The surface areas of the two pads are typically, but not necessarily, the same.) Alternatively or additionally, a length of each of adhesive pads 26 is less than 10 cm. The adhesive sides of adhesive pads 26 may be protected with respective removable peel-off backings 21, as is known in the adhesive arts. In general, all of the adhesive pads described herein are provided with a sufficient and appropriate size to hold the oxygen mask, tracheostomy-tube holder, or cannula in such a way to ensure proper placement for optimal delivery while accounting for the anticipated movement for the subject.

Although apparatus 20 is generally described herein as being used for coupling an oxygen mask to the subject's face, apparatus 20 may also be used for coupling other kinds of masks to the subject's face, such as a filter mask.

A plurality of (typically exactly two) straps 28 (e.g., elastic straps 28) are coupleable to respective adhesive pads 26 at respective first ends 23 of the straps. Respective second ends 25 of straps 28, opposite the first ends, are coupleable to mask 22. To this end, for some applications, second ends 25 of straps 28 are shaped so as to define respective rotation couplings 37, which are rotatably coupleable to respective coupling holes 41 defined by mask 22, typically so as to allow 360 degrees of rotation of second ends 25 of straps 28 with respect to respective coupling holes 41.

FIG. 1A shows straps 28 before being coupled to oxygen mask 22 and adhesive pads 26, and FIG. 1B shows the straps coupled to the oxygen mask and the adhesive pads, respectively. Typically, rotation couplings 37 are rotated before, and/or after, and/or while adhering adhesive pads 26 to the face of the subject. The rotation of rotation couplings 37 generally reduces the mechanical forces (e.g., pressure, tension, or rubbing) applied by the apparatus to the skin of the subject.

For some applications, rotation couplings 37 are shaped so as to define respective head portions 43 and respective neck portions 45 that are adjacent to and narrower than the head portions. Head portions 43 are configured to snap through coupling holes 41, such that neck portions 45 reside within and pass through the coupling holes. For example, head portions 43 may be generally bulbous, generally spherical, generally trapezoidal in cross-section, generally triangular in cross-section, or generally rectangular in cross-section. Typically, each of head portions 43 has a volume of at least 0.5 cm3, no more than 1.5 cm3, and/or between 0.5 cm3 and 1.5 cm3. Typically, each of neck portions 45 has a length of at least 1 mm, no more than 7 mm (e.g., no more than 5 mm), and/or between 1 and 7 mm, e.g., between 1 and 5 mm, and/or an average diameter of at least 2 mm, no more than 9 mm, and/or between 2 and 9 mm.

For some applications, each of straps 28 is coupleable to one of adhesive pads 26 so as to provide an adjustable total length of the combined strap and adhesive pad, i.e., an adjustable total distance between second end 25 of strap 28 and a far end 51 of adhesive pad 26 (i.e., the end of the pad opposite the end of the pad that is coupleable to the strap). For example, the end of the adhesive pad that is coupleable to the strap may be shaped so as to define a tab 29, which is sized so as to be passable through a slit 47 defined by strap 28 near first end 23 of strap 28 (e.g., within 10 mm of first end 23). Tab 29 and pad 26 are configured such that the tab is fastenable to another portion of the pad. For example, the tab and/or the pad may comprise a hook-and-loop fastener 53 (as shown), a snap-fastener, a button-and-buttonhole, an adhesive, and/or a protrusion-coupler. The combined length may be adjusted before and/or after the pads have been adhered to the face, and before and/or after the straps have been coupled to the mask.

FIG. 2 shows oxygen mask 22 coupled to the subject's face by adhesive pads 26 and straps 28; the adhesive pads adhere to respective cheeks of the subject. Typically, oxygen mask 22 is conventional, and defines holes 41 for the conventional use of attaching conventional head straps to the mask; in the present application of the present invention, these conventional holes are instead used for coupling to rotation couplings 37. Typically, each strap 28 has a length L that is at least 2 cm (e.g., at least 5 cm) and/or less than 40 cm (e.g., less than 15 cm), and/or between 2 and 40 cm, e.g., between 5 and 15 cm. In order to provide the adherence with skin of the cheeks, the adhesive pads comprise an adhesive formulated for adhering to the skin.

Reference is again made to FIG. 2. To couple oxygen mask 22 to the subject's face, adhesive pads 26 are typically first adhered to a surface of the subject's face, such as respective cheeks of the subject, typically without adhering the adhesive pads to the subject's nose. Subsequently to the adhering of the pads, straps 28 are coupled to coupling holes 41 of the oxygen mask, such as described hereinabove. Straps 28 may be attached to adhesive pads 26 before or after adhering the pads to the face, and before or after coupling the straps to the oxygen mask. For applications in which straps 28 are coupleable to adhesive pads 26 so as to provide an adjustable total length of the combined straps and adhesive pads, such as described above, the combined length may be adjusted before and/or after the pads have been adhered to the face, and before and/or after the straps have been coupled to the oxygen mask. For some applications, oxygen mask 22 is coupled to the subject's face such that no portion of the mask touches a nose of the subject.

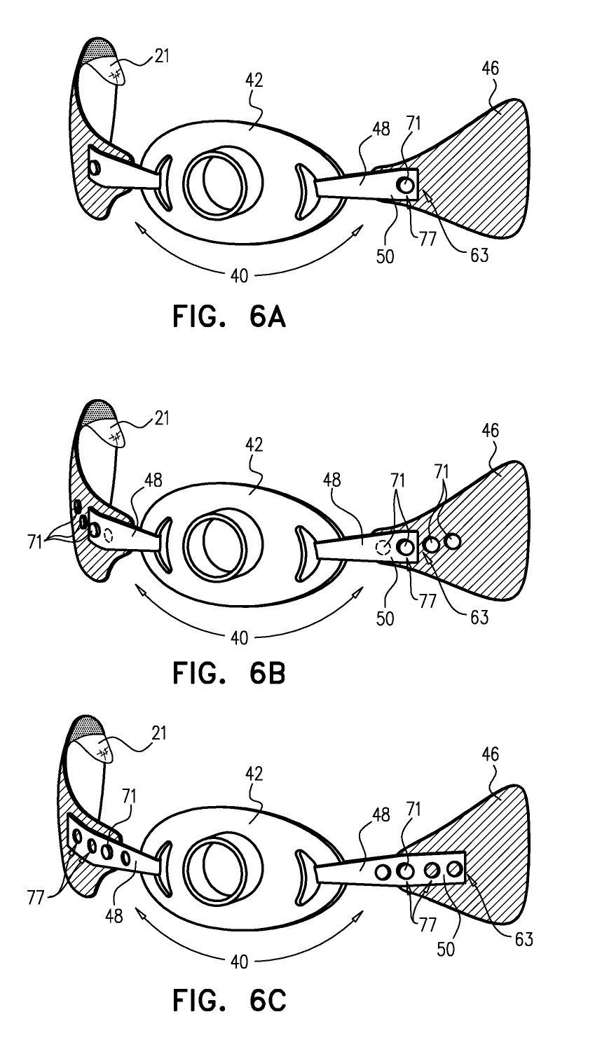

Reference is made to FIG. 3, which is a schematic illustration of an attachment of strap 28 to adhesive pad 26, in accordance with an application of the present invention. In this configuration, straps 28 are rotatably coupled to respective adhesive pads 26. For example, apparatus 20 may comprise a second rotation coupling 49 for each pair of straps 28 and adhesive pads 26. Second rotation coupling 49 couples strap 28 to adhesive pad 26 near first end 23 of strap 28, so as to allow rotation of strap 28 with respect to adhesive pad 26. Typically, second rotation couplings 49 are rotated before, and/or after, and/or while adhering adhesive pads 26 to the face of the subject. The rotation of rotation coupling 49 generally reduces the mechanical forces (e.g., pressure, tension, or rubbing) applied by the apparatus to the skin of the subject. For some applications, straps 28 and adhesive pads 26 implement one or more of the coupling techniques described hereinbelow with reference to FIGS. 6A-C, such as in order to provide an adjustable total length of the combined strap and adhesive pad.

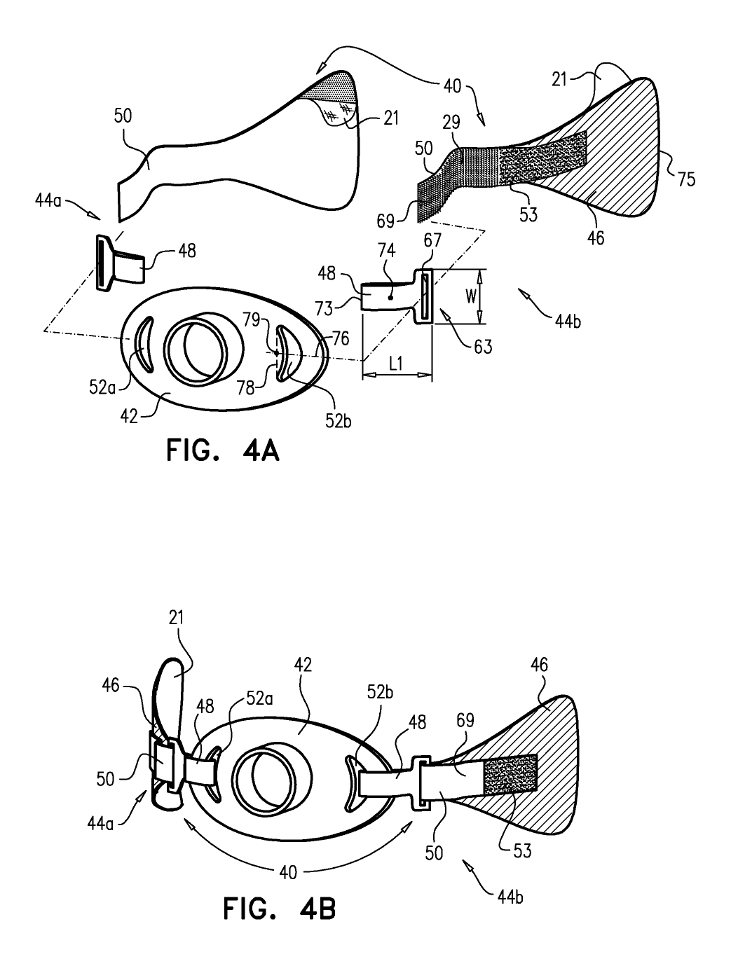

In some applications, each of straps 28 comprises distal and proximal portions, which are provided (e.g., in sterile packaging) as separate elements that are not coupled together. Typically, distal portion of the strap is attachable to proximal portion of the strap even without being tied to the proximal portion. For example, apparatus 20 may comprise a coupler, and the distal portion of the strap is attachable to the proximal portion of the strap via the coupler. The coupler may comprise, for example, a hook-and-loop, a snap-fastener, a button-and-buttonhole, an adhesive, and/or a protrusion-coupler. (In a protrusion coupler, one or more protrusions on one of the portions of the strap pass through an opening on the other one of the portions.) These applications may be implemented as shown in FIG. 3 of above-mentioned U.S. Provisional Application 62/044,622, mutatis mutandis, in which the distal portion is labeled with the reference numeral 34 and the proximal portion is labeled with the reference numeral 36. In some applications, the distal portion of the strap is attachable to the proximal portion of the strap via a clipping action, a clamping action, and/or a stapling action. Examples of couplers that facilitate these actions, including a clip, a clamp, and a stapler, are shown in FIG. 4 of above-mentioned U.S. Provisional Application 62/044,622.

In accordance with some of these applications, apparatus 20 is provided for coupling oxygen mask 22 to a face of a subject, oxygen mask 22 shaped so as to define two coupling holes 41, the apparatus comprising: (a) two adhesive pads 26 configured to adhere to a surface of the subject's face, each of the 26 pads having a surface area between 1 and 50 cm2; and (b) two straps 28. Each of straps 28 (i) comprises distal and proximal portions, which are provided as separate elements that are not coupled together, and which are configured to be attachable to each other without being tied, and (ii) has a length, when the distal and the proximal portions are attached together, of between 2 and 40 cm. The respective proximal portions of straps 28 are coupleable to adhesive pads 26, respectively. The respective distal portions of straps 28 are shaped so as to define respective rotation couplings 37, which are rotatably coupleable to coupling holes 41 of oxygen mask 22, respectively. These techniques may be practiced in combination with any of the other techniques described herein with reference to FIGS. 1-3 hereof, mutatis mutandis.

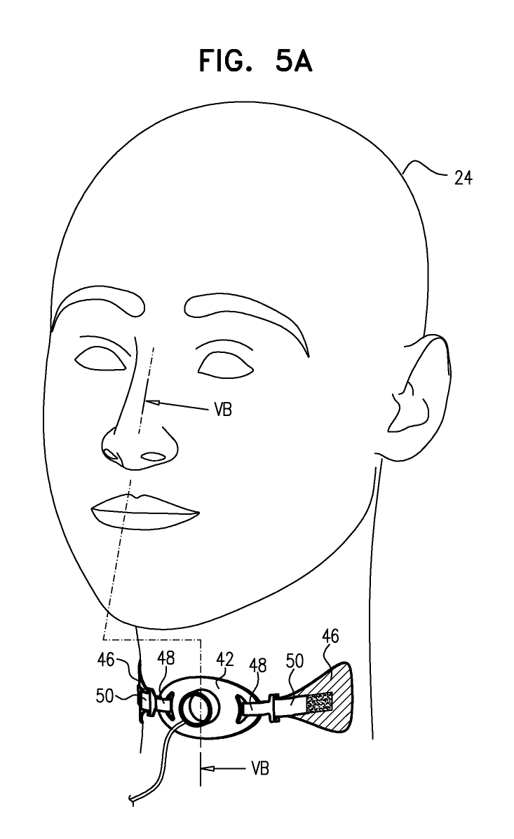

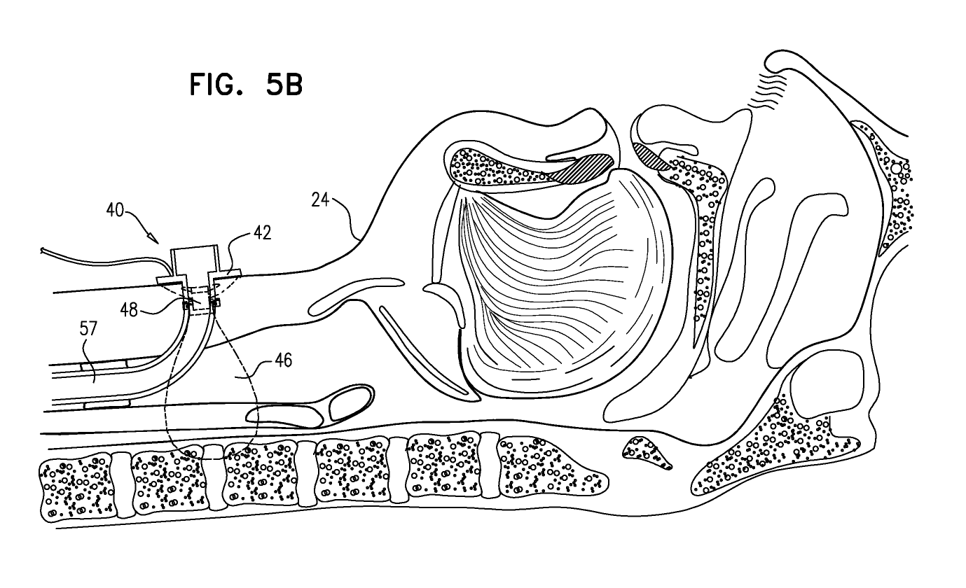

Reference is now made to FIGS. 4A-B, which are schematic illustrations of apparatus 40 for supporting a tracheostomy-tube holder 42 over a tracheostomy of subject 24, and to FIGS. 5A-B, which are schematic illustrations of apparatus 40 coupled to the neck of the subject, in accordance with some applications of the present invention. Apparatus 40 comprises a first support 44a and a second support 44b. Each of supports 44a and 44b comprises an adhesive pad 46, a strap 48, and a strap-fastener 50. Each of strap-fasteners 50 is coupled to an adhesive pad 46, and is also coupleable to a proximal portion of strap 48. FIG. 4A shows straps 48 before being coupled to tracheostomy-tube holder 42 and adhesive pads 46, and FIG. 4B shows the straps coupled to the tracheostomy-tube holder and the adhesive pads. The adhesive sides of adhesive pads 46 may be protected with respective removable peel-off backings 21, as is known in the adhesive arts.

Typically, a tracheostomy tube 57 is inserted into the subject's trachea (as shown in FIG. 5B), and thereafter tracheostomy-tube holder 42 is connected to tracheostomy tube 57 and brought into contact with the subject's neck, so as to support tracheostomy tube 57 within the subject's trachea. As shown in FIGS. 5A-B, to support tracheostomy-tube holder 42, adhesive pads 46 are adhered to the neck of the subject, either before or after connecting tracheostomy-tube holder 42 to tracheostomy tube 57, and respective straps 48 are passed through a first slot 52a and a second slot 52b of tracheostomy-tube holder 42, either before or after connecting tracheostomy-tube holder 42 to tracheostomy tube 57. Straps 48 may be attached to adhesive pads 46 before or after adhering the pads to the face, and before or after coupling the straps to tracheostomy-tube holder 42. For applications in which straps 48 are coupleable to adhesive pads 46 so as to provide an adjustable total length of the combined straps and adhesive pads, such as described below, the combined length may be adjusted before and/or after the pads have been adhered to the neck, and before and/or after the straps have been coupled to the tracheostomy-tube holder.

Typically, respective straps 48 pass through first and second slots 52a and 52b of tracheostomy-tube holder 42 so as to provide leeway for the straps to move with respect to the tracheostomy-tube holder, so as to reduces the mechanical forces (e.g., pressure, tension, or rubbing) applied by the apparatus to the skin of the subject. Typically, each of first and second slots 52a and 52b has a longer and a shorter dimension. For some applications, in order to provide such leeway, when each of straps 48 are coupled to one of first and second slots 52a and 52b, (a) a first line 76 (i) is perpendicular with a line segment 78 between the ends of the longer dimension of the slot, and (ii) intersects a midpoint 79 of line segment 78, and (b) the strap is moveable with respect to the tracheostomy-tube holder to an extent such that a second line between midpoint 79 and a geometric center point 74 of the strap can form an angle of at least 15 degrees with first line 76. (It is to be understood that the first and second lines and midpoint are not elements of the invention, but are rather geometric shapes used to describe structural characteristics of the apparatus.) Alternatively or additionally, for some applications, in order to provide such leeway, when respective straps 48 are coupled to first and second slots 52a and 52b, each of the straps can move, with respect to tracheostomy-tube holder 42, along the long dimension of the slot by at least 7.5 mm, and, optionally, can move along the short dimension of the slot by at least 5 mm.

Typically, each of straps 48 has a length L1 that is at least 1 cm (e.g., at least 5 cm) and/or less than 40 cm (e.g., no more than 20 cm), and/or between 1 and 40 cm, e.g., between 5 and 20 cm, and a greatest width W that is at least 0.25 cm (e.g., at least 0.5 cm) and/or less than 3 cm (e.g., no more than 2 cm), e.g., between 0.5 and 2 cm. (The lengths of the straps are typically the same, and the widths of the straps are typically the same.) Typically, the adhesive pads have respective surface areas that are at least 5 cm2 and/or less than 15 cm2. (The surface areas of the two adhesive pads are typically the same.) A relatively short length L1, and/or relatively small surface areas, are typically appropriate for pediatric subjects, while a relatively large L1, and/or relatively large surface areas, are typically appropriate for large subjects.

For some applications, each of straps 48 is coupleable to one of adhesive pads 46 so as to provide an adjustable total length of the combined strap and adhesive pad, i.e., an adjustable total distance between a far end 73 of strap 48 and a far end 75 of adhesive pad 46 (i.e., the end of the pad opposite the end of the pad that is coupleable to the strap). For example, strap-fastener 50 may be shaped so as to define a tab 69, which is sized so as to be passable through a slit 67 defined by strap 48 near a first end 63 of strap 48 (e.g., within 10 mm of first end 63) (first end 63 is the end of strap 48 that is coupled to pad 46). Tab 69 and pad 46 are configured such that the tab is fastenable to another portion of the pad. For example, the tab and/or the pad may comprise a hook-and-loop fastener 53, a snap-fastener, a button-and-buttonhole, an adhesive, and/or a protrusion-coupler.

Reference is made to FIGS. 6A-C, which are schematic illustrations of attachment of strap 48 to adhesive pad 46, in accordance with respective applications of the present invention. In these configurations, straps 48 are rotatably coupled to adhesive pads 46, respectively. For example, apparatus 40 may comprise a rotation coupling 71 for each pair of straps 48 and adhesive pads 46. Rotation coupling 71 couples strap 48 to adhesive pad 46 near first end 63 of strap 48, so as to allow rotation of strap 48 with respect to adhesive pad 46. Typically, rotation couplings 71 are rotated before, and/or after, and/or while adhering adhesive pads 46 to the neck of the subject. The rotation of rotation couplings 71 generally reduces the mechanical forces (e.g., pressure, tension, or rubbing) applied by the apparatus to the skin of the subject. For some applications, rotational couplings 71 fixedly rotationally couple straps 48 to respective adhesive pads 46.

For some applications, rotational couplings 71 are configured to be coupled during application of apparatus 40 to the subject. For example, rotational couplings 71 may be fixed to respective adhesive pads 46, and configured to be coupled to respective straps 48. For example, each of adhesive pads 46 may be shaped to define one or more holes 77, and each of rotational couplings 71 may be configured to snap into one of holes 77. For some of these applications, such as shown in FIG. 6A, each of straps 48 is shaped so as to define a single hole 77, and each of adhesive pads 46 comprises a single rotational coupling 71. For others of these applications, such as shown in FIGS. 6B-C, each of straps 48 are coupleable to one of adhesive pads 46 so as to provide an adjustable total length of the combined strap and adhesive pad. For example, as shown in FIG. 6B, each of adhesive pads 46 may comprise a plurality of rotational couplings 71 distributed along the pad, and each of straps 48 may be shaped so as to define a single hole 77. The selected rotational coupling sets the total length of the combined strap and adhesive pad. Alternatively, as shown in FIG. 6C, each of straps 48 may be shaped so as to define a plurality of holes 77 distributed along the strap, and each of adhesive pads 46 may comprise a single rotational coupling 71. The selected hole 77 sets the total length of the combined strap and adhesive pad. Further alternatively, each of adhesive pads 46 may comprise a plurality of rotational couplings 71 distributed along the pad, and each of straps 48 may be shaped so as to define a plurality of holes 77 distributed along the strap (configuration not shown).

For some applications, each of straps 48 comprises distal and proximal portions 54 and 56, which are provided (e.g., in sterile packaging) as separate elements that are not coupled together. Typically, respective distal portions 54 of straps 48 are attachable to respective proximal portions 56 of the straps, even without being tied to the proximal portions. For example, apparatus 40 may comprise first and second couplers, and the respective distal portions of the straps are attachable to the respective proximal portions of the straps via respective couplers. Each of the couplers may comprise a hook-and-loop, a snap-fastener, a button-and-buttonhole, an adhesive, and/or a protrusion-coupler. These applications may be implemented as shown in FIG. 3 of above-mentioned U.S. Provisional Application 62/044,622, mutatis mutandis. Alternatively or additionally to the use of the couplers, the respective distal portions of the straps may be attachable to the respective proximal portions of the straps via clipping, clamping, and/or stapling, as described hereinabove with reference to FIG. 4 of above-mentioned U.S. Provisional Application 62/044,622.

In accordance with some of these applications, apparatus 40 for supporting tracheostomy-tube holder 42 over a tracheostomy, the apparatus comprising first and second supports 44a and 44b, comprising (a) respective adhesive pads 46; and (b) respective straps 48. Each of straps 48 (i) comprises distal and proximal portions, which are provided as separate elements that are not coupled together, and which are configured to be attachable to each other without being tied, and (ii) has a length, when the distal and the proximal portions are attached together, of between 1 and 40 cm and a width between 0.25 and 3 cm. The respective proximal portions of straps 48 are configured to be coupled to adhesive pads 46, respectively. The respective distal portions of straps 48 are configured to be coupled to tracheostomy-tube holder 42. These techniques may be practiced in combination with any of the other techniques described herein with reference to FIGS. 4A-6C hereof, mutatis mutandis.

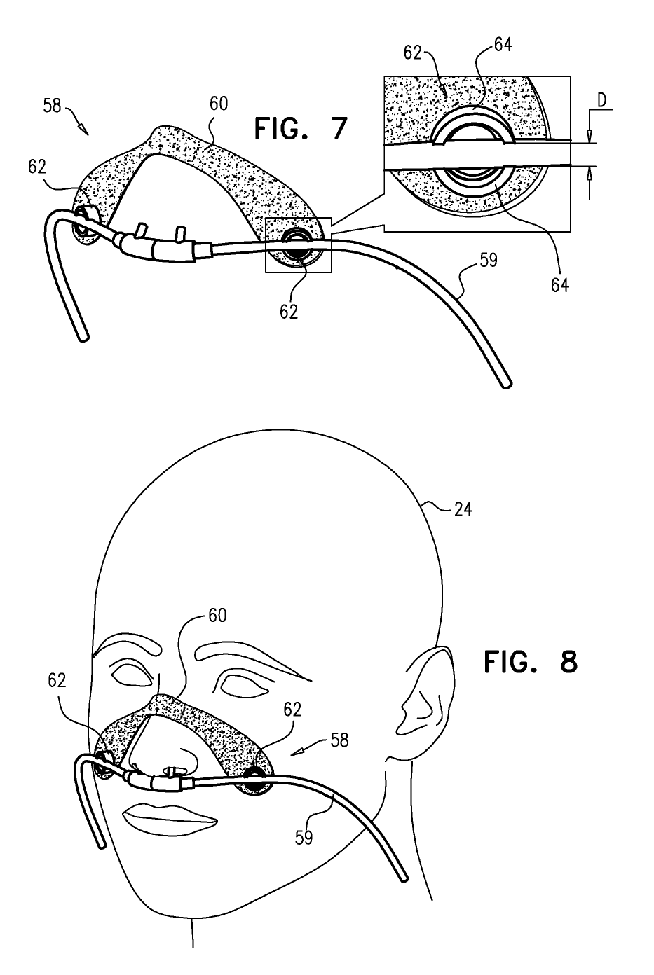

Reference is now made to FIG. 7, which is a schematic illustration of apparatus 58 for coupling a cannula 59 to a face of subject 24, in accordance with some applications of the present invention. Apparatus 58 comprises an adhesive pad 60 configured to adhere to a surface of the subject's face. Typically, adhesive pad 60 has a surface area A4 that is at least 2 cm2 and/or less than 10 cm2, e.g., between 2 and 5 cm2. At least one cannula grip 62 is rotatably coupled (typically fixed) to pad 60, and is configured to couple cannula 59 to pad 60 and thus to the subject's face by gripping the cannula. For example, as shown in FIG. 7, two cannula grips 62 may be rotatably coupled (typically fixed) to respective portions of the adhesive pad. The adhesive side of adhesive pad 60 may be protected with removable peel-off backings, as is known in the adhesive arts.

To couple cannula 59 to the subject's face, adhesive pad 60 is adhered to a surface of the subject's face, and the cannula is (typically subsequently) gripped with cannula grip 62 such that the cannula is coupled to adhesive pad 60. Typically, cannula grip 62 is rotated before, after, and/or while cannula 59 is coupled to adhesive pad 60 by gripping. The rotation of cannula grip 62 generally facilitates an effective gripping of the cannula, and/or reduces the mechanical forces (e.g., pressure, tension, or rubbing) applied by the apparatus to the skin of the subject. The coupling of cannula 59 to the subject's face, as described herein, may be practiced in combination with techniques described in U.S. Pat. No. 6,328,038 to Kessler (e.g., with reference to FIG. 2 thereof), which is incorporated herein by reference. Typically, each cannula grip 62 is shaped to define two gripping arms 64 configured to grip cannula 59 therebetween, a closest distance D between the gripping arms being between 2 and 10 mm.

Typically, adhesive pad 60 is elongate, and has a length of at least 1 cm, no more than 10 cm, and/or between 1 and 10 cm, a smallest width of at least 0.3 cm, no more than 2 cm, and/or between 0.3 and 2 cm, and a greatest width of at least 0.5 cm, no more than 3 cm, and/or between 0.5 and 3 cm. Typically, the length of adhesive pad 60 equals at least 100%, no more than 1000%, and/or between 100% and 1000% of the greatest width, and/or at least 100%, no more than 2000%, and/or between 100% and 2000% of the smallest width. For some applications, widest portions of the adhesive pad are near respective end portions of the adhesive pad, and a narrowest portion of the adhesive pad is at or near the longitudinal center of the pad.

Typically, adhesive pad 60 is elongate (such as described above), and cannula grips 62 are disposed on respective end portions of adhesive pad 60, i.e., near respective ends of adhesive pad, e.g., within 1 cm (e.g., 0.5 cm) of the respective ends (i.e., the closest portions of the grips are within 1 cm (e.g., 0.5 cm) mm of the respective ends).

Reference is now made to FIG. 8, which is a schematic illustration of apparatus 58 attached to the face of subject 24, in accordance with an application of the present invention. In this configuration, adhesive pad 60 is adhered to a surface of a nose of the subject and to the cheeks of the subject; exactly one adhesive pad, such as the pad shown in FIG. 7, is typically used. In this configuration, adhesive pad 60 is disposed such that a central portion of the pad is adhered to the nose, and end portions of adhesive pad 60, which include cannula grips 62, respectively, are adhered to respective cheeks of the subject.

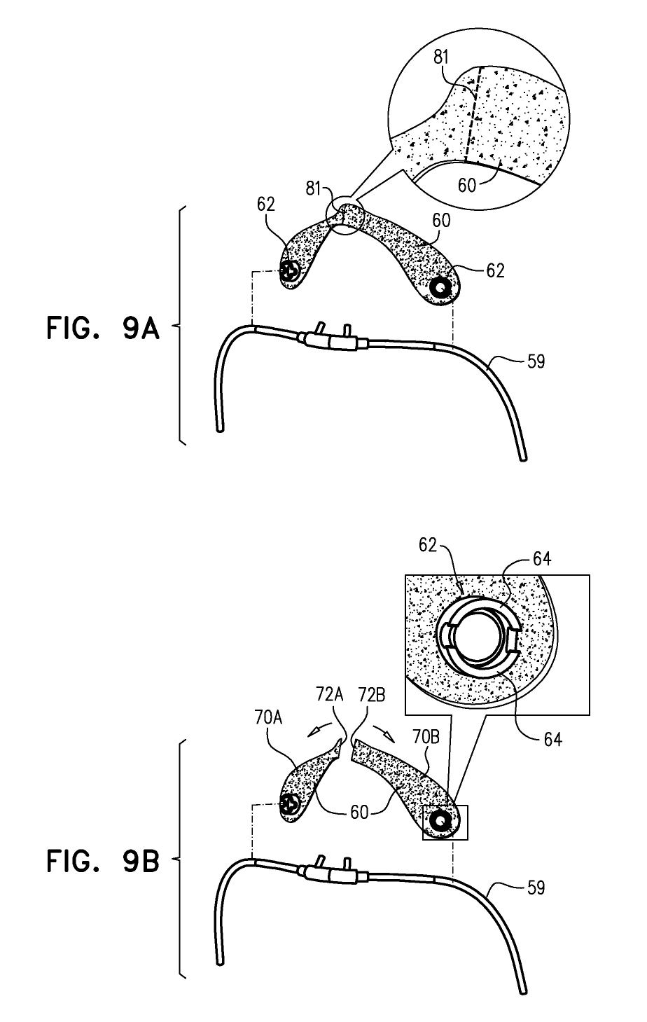

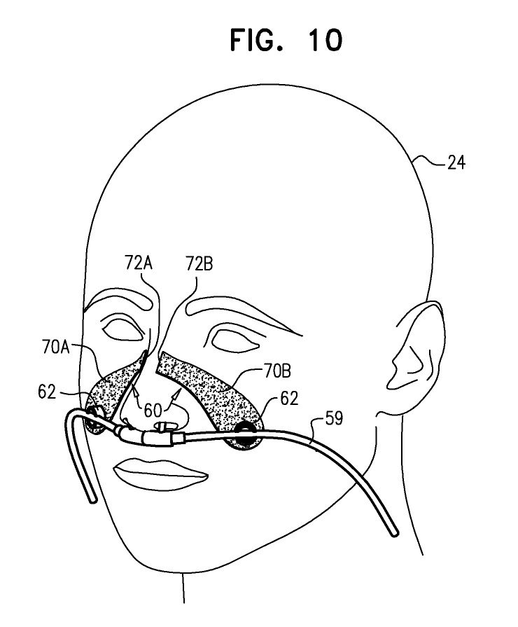

Reference is now made to FIGS. 9A-B, which are schematic illustrations of splitting of adhesive pad 60 into two pieces 70A and 70B, in accordance with an application of the present invention. In this application, before adhesive pad 60 is adhered to the surface of the subject's face, the adhesive pad is split into two pieces 70A and 70B. The two pieces are adhered at least partially to respective cheeks of the subject. For some applications, such as shown in FIG. 9A, adhesive pad 60 is shaped so as to define a weakened line 81 to facilitate such splitting (in which case the pad is split along weakened line 81). For example, weakened line 81 may be perforated, scored, or indented. (Weakened line 81 is not necessarily straight.) Providing adhesive pad 60 as a single piece with weakened line 81 provides the option to either attach the pad to the face as a single piece, such as shown in FIG. 8, or to split the pad and attach it to the face as two pieces, such as shown in FIGS. 10 and 12. For other applications the adhesive pad is cut into two pieces, such as using scissors (for example, the pad may include a visual indicator, such as a line (which is not necessarily straight), demarcating the pad into the two pieces, and indicating a suggested site for cutting). The splitting the pad into two pieces, whether by separating along weakened line 81 or by cutting, allows adhesive pad 60 to accommodate different sized noses, without the need to provide different sized pads for different sized noses. In other words, a single pad size can accommodate a plurality of different sized noses.

Weakened line 81 or the visual indicator is typically disposed at a longitudinal center of the pad, crossing the width of the pad, such that the pad is split into two equally-sized pieces 70A and 70B. Pieces 70A and 70B typically have respective surface areas A5 that are greater than 0.5 cm2 and/or less than 10 cm2, and/or between 0.5 and 10 cm2. Typically, adhesive pad 60 is provided (e.g., in sterile packaging) as a single piece, i.e., before it is split into two pieces.

For still other applications, two separate adhesive pads are provided (e.g., in sterile packaging), which are configured to adhere to respective cheeks of the subject. For example, the two pads may have respective surface areas that are less than 10 cm2. The two pads may have any of the features of pieces 70A and 70B, respectively, described hereinabove and/or show in the figures.

Reference is made to FIG. 10, which is a schematic illustration of pieces 70A and 70B adhered to a surface of the subject's face, in accordance with an application of the present invention. In this configuration, pieces 70A and 70B, as they are adhered to the face, are maintained generally in the same rotational relationship with each other after splitting the pad into the two pieces, such that respective portions of the two pieces are adhered to respective sides of the nose (with split edges 72A and 72B facing each other), and other respective end portions of the two pieces, which include cannula grips 62, respectively, are adhered to respective cheeks of the subject. (Split edges 72A and 72B are the edges that are created by splitting the adhesive pad.)

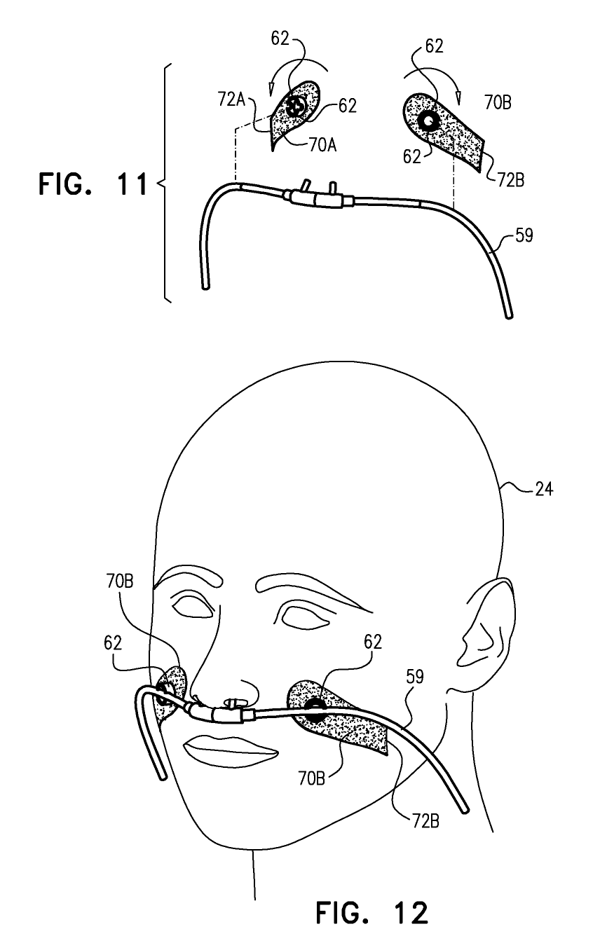

Reference is made to FIG. 11, which is a schematic illustration of rotation of pieces 70A and 70B, in accordance with an application of the present invention. In this configuration, after adhesive pad 60 has been split into two pieces 70A and 70B, such as described hereinabove with reference to FIGS. 7A-B, the pieces are rotated (typically generally about cannula grips 62) until respective split edges 72A and 72B of pieces 70A and 70B face generally away from each other.

Reference is made to FIG. 12, which is a schematic illustration of pieces 70A and 70B adhered to a surface of the subject's face after being rotated as described with reference to FIG. 11, in accordance with an application of the present invention. Pieces 70A and 70B are adhered to respective cheeks of the subject, typically without being adhered to the subject's nose, and typically with respective split edges 72A and 72B pointing generally away from the nose.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for coupling a cannula to a face of a subject, the apparatus comprising:
an adhesive pad configured to adhere to a surface of the subject's face; and
one or more cannula grips rotatably coupled to the adhesive pad and configured to couple the cannula to the subject's face by gripping the cannula.

2. The apparatus according to claim 1, wherein each of the one or more cannula grips is shaped to define two gripping arms configured to grip the cannula therebetween, a distance between the gripping arms being between 2 and 10 mm.

3. The apparatus according to claim 1, further comprising the cannula.

4. The apparatus according to claim 1, wherein the adhesive pad has a surface area between 2 and 10 cm2.

5. (canceled)

6. The apparatus according to claim 1, wherein the apparatus comprises two adhesive pads, which are configured to adhere to respective cheeks of the subject, and which have respective surface areas that are less than 10 cm2.

7. The apparatus according to claim 1, wherein the apparatus comprises two cannula grips, which are rotatably coupled to the adhesive pad and configured to couple the cannula to the subject's face by gripping the cannula.

8. The apparatus according to claim 7,
wherein the adhesive pad is shaped so as to define a weakened line to facilitate splitting of the adhesive pad into two pieces, which are configured to adhere to respective cheeks of the subject, and
wherein the two cannula grips are rotatably coupled to the two pieces, respectively.

9. The apparatus according to claim 8, wherein the weakened line is perforated.

10. The apparatus according to claim 7,
wherein the adhesive pad comprises a visual indicator demarcating the adhesive pad into two pieces, which are configured to adhere to respective cheeks of the subject, and
wherein the two cannula grips are rotatably coupled to the two pieces, respectively.

11. The apparatus according to claim 7,
wherein the apparatus comprises two adhesive pads, which are configured to adhere to respective cheeks of the subject, and which have respective surface areas that are less than 10 cm2, and
wherein the two cannula grips are rotatably coupled to the two adhesive pads, respectively.

12. A method for coupling a cannula to a face of a subject, the method comprising:
adhering an adhesive pad to a surface of the subject's face; and
coupling the cannula to the subject's face by gripping the cannula with one or more cannula grips that are rotatably coupled to the adhesive pad.

13. The method according to claim 12, wherein each of the one or more cannula grips is shaped to define two gripping arms configured to grip the cannula therebetween, a distance between the gripping arms being between 2 and 10 mm, and wherein coupling the cannula to the subject's face comprises gripping the cannula with the gripping arms.

14. The method according to claim 12, wherein the adhesive pad has a surface area between 2 and 10 cm2.

15. (canceled)

16. The method according to claim 12, wherein gripping the cannula with the one or more cannula grips comprises gripping the cannula with the one or more cannula grips while rotating the one or more cannula grips.

17-18. (canceled)

19. The method according to claim 12, wherein coupling the cannula to the subject's face comprises coupling the cannula to the subject's face after adhering the adhesive pad to the surface of the subject's face.

20. The method according to claim 12, wherein adhering the adhesive pad to the surface of the subject's face comprises adhering the adhesive pad to a surface of a nose of the subject.

21. The method according to claim 20, wherein adhering the adhesive pad to a surface of a nose of the subject comprises adhering exactly one adhesive pad to the surface of the nose of the subject.

22. The method according to claim 21,
wherein the one or more cannula grips comprise two cannula grips, which are rotatably coupled to the adhesive pad and configured to couple the cannula to the subject's face by gripping the cannula, and
wherein adhering the exactly one adhesive pad to the surface of the subject's face comprises:
adhering a central portion of the exactly one adhesive pad to the surface of the nose; and
adhering end portions of the exactly one adhesive pad to respective cheeks of the subject, wherein the end portions include the two cannula grips, respectively.

23. The method according to claim 12, wherein adhering the adhesive pad to the surface of the subject's face comprises adhering two adhesive pads to respective cheeks of the subject, without adhering the adhesive pads to a nose of the subject.

24. (canceled)

25. The method according to claim 12, wherein coupling the cannula to the subject's face comprises gripping the cannula with two cannula grips that are rotatably coupled to the adhesive pad.

26. The method according to claim 25, wherein adhering the adhesive pad to the surface of the subject's face comprises:
splitting the adhesive pad into two pieces, wherein the two cannula grips are rotatably coupled to the two pieces, respectively; and
adhering the two pieces at least partially to respective cheeks of the subject.

27. The method according to claim 26, wherein adhering the two pieces at least partially to respective cheeks of the subject comprises maintaining the two pieces generally in a same rotational relationship with each other after splitting the adhesive pad into the two pieces, such that respective portions of the two pieces are adhered to respective sides of a nose of the subject, and other respective end portions of the two pieces, which include the two cannula grips, respectively, are adhered to the respective cheeks of the subject.

28. The method according to claim 26, wherein adhering the two pieces at least partially to respective cheeks of the subject comprises rotating the two pieces until respective split edges of the two pieces face generally away from each other, and subsequently adhering the two pieces to the respective cheeks of the subject, without adhering the two pieces to a nose of the subject.

29. The method according to claim 26, wherein the adhesive pad is shaped so as to define a weakened line to facilitate splitting of the adhesive pad into two pieces, and wherein splitting the adhesive pad into the two pieces comprises splitting the adhesive pad into the two pieces along the weakened line.

30. The method according to claim 29, wherein the weakened line is perforated.

31. The method according to claim 26, wherein splitting the adhesive pad into the two pieces comprises cutting the adhesive pad into the two pieces.

32. The method according to claim 31, wherein the adhesive pad comprises a visual indicator demarcating the adhesive pad into the two pieces, and wherein splitting the adhesive pad into the two pieces comprises cutting the adhesive pad along the visual indicator.

33. The method according to claim 25, wherein adhering the adhesive pad to the surface of the subject's face comprises adhering two adhesive pads to respective cheeks of the subject, wherein the two adhesive pads have respective surface areas that are less than 10 cm2, and wherein the two cannula grips are rotatably coupled to the two adhesive pads, respectively.

34-84. (canceled)
